(19) United States
(12) Patent Application Publication
(10) Pub. No.: US 2014/0377319 A1
(43) Pub. Date: Dec. 25, 2014

(54) ALTERING PROTEIN CONCENTRATIONS IN CEREBROSPINAL FLUID AND/OR BRAIN INTERSTITIAL FLUID

(71) Applicants: Eric C. Leuthardt, St. Louis, MO (US); Daniel W. Moran, St. Louis, MO (US); David Holtzman, St. Louis, MO (US); Philip Bayly, St. Louis, MO (US); David Brody, St. Louis, MO (US); Ralph G. Dacey, St. Louis, MO (US); Sam Fok, St. Louis, MO (US); David Jeremiah Garrison, Charlottesville, VA (US); Guy M. Genin, St. Louis, MO (US); Eriks Lusis, St. Louis, MO (US); Rohit Pappu, St. Louis, MO (US); Jingwei Xie, Chesapeake, OH (US); Greg Zipfel, University City, MO (US)

(72) Inventors: Eric C. Leuthardt, St. Louis, MO (US); Daniel W. Moran, St. Louis, MO (US); David Holtzman, St. Louis, MO (US); Philip Bayly, St. Louis, MO (US); David Brody, St. Louis, MO (US); Ralph G. Dacey, St. Louis, MO (US); Sam Fok, St. Louis, MO (US); David Jeremiah Garrison, Charlottesville, VA (US); Guy M. Genin, St. Louis, MO (US); Eriks Lusis, St. Louis, MO (US); Rohit Pappu, St. Louis, MO (US); Jingwei Xie, Chesapeake, OH (US); Greg Zipfel, University City, MO (US)

(73) Assignee: WASHINGTON UNIVERSITY, St. Louis, MO (US)

(21) Appl. No.: 14/112,817

(22) PCT Filed: Apr. 18, 2012
(86) PCT No.: PCT/US12/34120

§ 371 Date: Apr. 25, 2014

Related U.S. Application Data
(60) Provisional application No. 61/477,839, filed on Apr. 21, 2011.

(51) Int. Cl. A61K 38/48; A61K 9/00; A61K 47/48; C12N 11/08

(52) U.S. Cl. CPC A61K 38/4886; C12N 11/08; A61K 9/0085; A61K 47/482; C12Y 304/24011
USPC 424/422; 435/180; 435/268; 424/94.67

(57) ABSTRACT

Devices and methods of altering protein concentrations in cerebrospinal fluid and/or brain interstitial fluid are disclosed. Devices include a support having at least one protease attached to the support. The device may further include a housing. Devices may be implantable for use in an in vivo active flow system or for use in an in vivo passive system. Devices may also be used in an ex vivo active flow system. Devices may also be used in a passive system to treat cerebrospinal fluid and/or brain interstitial fluid that is withdrawn, or in a passive system that is implanted surgically. Methods include contacting cerebrospinal fluid and/or brain interstitial fluid with a support including at least one protease attached to the support. Proteins contained in the cerebrospinal fluid and/or brain interstitial fluid are cleaved by the protease resulting in the reduction of protein in cerebrospinal fluid and/or brain interstitial fluid. Additionally, methods are disclosed for treating neurodegenerative diseases and neurological disorders.

### CROSS-REFERENCE TO RELATED APPLICATIONS

This application claims priority to International Application Number PCT/US2012/034120, filed on Apr. 18, 2012, and U.S. Provisional Patent Application No. 61/477,839, filed on Apr. 21, 2011, the disclosures of which are hereby expressly incorporated by reference in their entireties.

### BACKGROUND OF THE DISCLOSURE

The present disclosure relates generally to devices and methods for altering protein concentrations. More particularly, the present disclosure relates to devices useful for filtering, reacting with, or catalyzing reactions within cerebrospinal fluid and/or brain interstitial fluid for removing, cleaving, and/or altering concentrations and quantities of proteins in cerebrospinal fluid and/or brain interstitial fluid. The present invention is also related to methods for removing, cleaving, and/or altering concentrations of protein in cerebrospinal fluid and/or interstitial fluid by contacting cerebrospinal fluid and/or interstitial fluid with a device having a support and at least one protease attached to the support. The present invention is also related to methods for treating neurodegenerative diseases such as, for example, Alzheimer's disease, and for treating acute neurological disorders using a device having a support and at least one protease attached to the support that cleaves proteins.

Many neurological diseases and disorders arise from protein deposits within cerebrospinal fluid and fluid contained within the interstitial spaces of the brain (“interstitial fluid”). Neurological diseases and disorders involving protein deposits include, for example, Alzheimer's disease, Parkinson's disease, prion disease, polyglutamine disease, Tauopathy, and Familial amyotrophic lateral sclerosis.

Alzheimer's disease, for example, is a terminal illness characterized by the progressive loss of memory and cognitive function. About five hundred thousand people are newly diagnosed each year, and it is projected that approximately a million people will be diagnosed per year by the middle of the century.

The protein amyloid beta appears to be associated with Alzheimer's disease. According to the Amyloid Hypothesis, amyloid beta in cerebrospinal fluid aggregates into oligomers, then precipitates and aggregates into amyloid plaques. Amyloid plaques interrupt synapse connections and serve as a precursor to and possible trigger of tau protein hyperphosphorylation. Tau protein usually interacts with tubulin to stabilize microtubules in neurons, but its hyperphosphorylation leads to the formation of tau tangles that interfere with neuronal transport of vesicles and cell death.

There is no cure for Alzheimer's at this time. Available treatments seek to replace lost function and are effective in about half of the population in delaying the progression of Alzheimer's for up to six months. Five drugs have been approved by the Food and Drug Administration to treat the symptoms of the disease, but their effects are temporary and not universal to all patients. Moreover, these drugs do not slow the progression of the disease and do not prolong survival. Furthermore, these drugs often entail dangerous side effects and unintended consequences.

Another approach for treating Alzheimer's disease uses size-exclusion filters. In this approach, cerebrospinal fluid is filtered by a membrane with a particular molecular weight cut-off that allows cerebrospinal fluid to pass through the membrane while trapping amyloid beta. While effective for removing amyloid beta from the cerebrospinal fluid, the filtering approach suffers from the disadvantage of the need for filter replacement because of protein build up on the filter.

Another approach for treating Alzheimer's disease involves adapting standard ventricular shunts to discharge cerebrospinal fluid into the peritoneal cavity, thereby transferring a fraction of the amyloid beta protein in the CSF out of the ventricles. This approach suffers the disadvantage of being limited by the capacity of the brain to produce cerebrospinal fluid, in that the maximum quantity of amyloid beta protein that can be removed is limited to that contained in the fraction of cerebrospinal fluid that can be safely shunted to the peritoneal cavity.

Prion diseases, for example, are another neurological disease that affects the structure of the brain and other neural tissue, which is currently untreatable. Mammalian prion diseases are caused by the prion protein, PrP. Aggregates of the abnormal PrP isoforms form highly structured amyloid fibers, which can accumulate to form plaques.

Although treatments are available for neurological diseases and disorders, their effects are temporary and not universal to all patients. Accordingly, there exists a need to develop alternative treatments and methods for treating neurological diseases and disorders.

### SUMMARY OF THE DISCLOSURE

The present disclosure relates generally to devices and methods for altering protein concentrations. More particularly, the present disclosure relates to devices useful for altering protein concentrations in cerebrospinal fluid and/or brain interstitial fluid and, potentially, plaques of these proteins. The present invention also relates to methods for altering protein in cerebrospinal fluid and/or brain interstitial fluid. The present invention also relates to methods for treating neurodegenerative diseases and acute neurological disorders by altering protein concentrations in cerebrospinal fluid and/or brain interstitial fluid.

In one aspect, the present disclosure is directed to a device for altering protein in cerebrospinal fluid and/or brain interstitial fluid. The device includes a support and at least one protease attached to the support, wherein the protease may be a zinc metalloprotease, a serine protease, trypsin, and pepsin.

In another aspect, the present disclosure is directed to a device for altering protein in cerebrospinal fluid and/or brain interstitial fluid. The device comprises: a support; and a protease attached to the support, wherein the protease is selected from the group consisting of a zinc metalloprotease, a serine protease, trypsin, pepsin, and combinations thereof.

In another aspect, the present disclosure is directed to methods for altering protein in cerebrospinal fluid and/or brain interstitial fluid. The method includes contacting cerebrospinal fluid with a device. The device includes a support and at least one protease attached to the support, wherein the at least one protease cleaves the protein.

In another aspect, the present disclosure is directed to a method for altering protein in a bodily fluid. The method comprises: contacting a bodily fluid selected from the group consisting of cerebrospinal fluid and brain interstitial fluid with a device, wherein the device comprises a support and a protease attached to the support, wherein the protease is selected from the group consisting of a zinc metalloprotease, a serine protease, trypsin, pepsin, and combinations thereof, and wherein the protease cleaves at least one protein in the bodily fluid.

In another aspect, the present disclosure is directed to a method for treating a neurological disease or disorder, the method comprising: contacting a bodily fluid selected from the group consisting of cerebrospinal fluid and brain interstitial fluid with a device, wherein the device comprises a support and a protease attached to the support, wherein the protease is selected from the group consisting of a zinc metalloprotease, a serine protease, trypsin, pepsin, and combinations thereof, and wherein the protease cleaves at least one protein in the bodily fluid.

In another aspect, the present disclosure is directed to methods for treating a neurodegenerative disease. The method includes contacting cerebrospinal fluid and/or brain interstitial fluid with a device. The device includes a support and at least one protease attached to the support.

In another aspect, the present disclosure is directed to methods for treating an acute neurological disorder. The method includes contacting cerebrospinal fluid and/or brain interstitial fluid with a device. The device includes a support and at least one protease attached to the support.

In another aspect, the present disclosure is directed to methods for treating Alzheimer's disease. The method includes contacting cerebrospinal fluid with a device. The device includes a support and at least one protease attached to the support, wherein the protease cleaves amyloid beta.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is an electrospinning apparatus for preparing nanofiber mats as described in Example 1.

FIG. 2 is a schematic illustrating EDC/NHS-mediated neprilysin attachment to air plasma treated poly(ε-caprolactone) fibers as described in Example 1.

FIG. 3 is a scanning electron micrograph of poly(ε-caprolactone) fibers before (A) and after (B) air plasma treatment as described in Example 1.

FIG. 4 is a silver-stained Tris-glycine gel showing the reduction of amyloid beta as described in Example 2.

FIG. 5 is a silver-stained Tris-glycine gel showing the reduction of amyloid beta as described in Example 3.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In accordance with the present disclosure, devices and methods have been discovered that allow for the alteration, cleavage, and/or reduction of protein concentrations in cerebrospinal fluid and/or brain interstitial fluid, and associated protein plaques. The devices and methods have significant impact as they allow for the prevention of the development of neurogenerative diseases and neurological disorders such as, for example, Alzheimer's disease, Parkinson's disease, Prion disease, Polyglutamine disease, Tauopathy, and Familial amyotrophic lateral sclerosis, systemic lupus erythematosus, and demylinating disorders, automimmune disorders, Gullain Barre Syndrome, and/or may reverse the progression of these diseases and disorders by altering, cleaving, and/or reduction of proteins in cerebrospinal fluid and/or brain interstitial fluid. The alteration, cleavage, and/or reduction of protein concentration in cerebrospinal fluid and/or brain interstitial fluid will delay or arrest the accumulation of proteins, protein oligomers, protein plaques, and protein deposits that further lead to cell death. The reduction of concentration within the cerebrospinal fluid and/or brain interstitial fluid may further lead to dissolution of amyloid beta protein oligomers and plaques, tau protein tangles, Lewy bodies, prion plaques, Bunina bodies, and other protein deposits.

### Compositions

In one aspect, the present disclosure is directed to a device for altering protein in cerebrospinal fluid and/or brain interstitial fluid including a support and at least one protease attached to the support. Proteases attached to the support cleave protein in cerebrospinal fluid resulting in the alteration and/or reduction of concentrations of proteins contained in cerebrospinal fluid and/or brain interstitial fluid.

Suitable proteases may be, for example, a zinc metalloprotease, a serine protease, trypsin, and pepsin. Suitable zinc metalloproteases may be, for example, insulin degrading enzyme (insulysin), presequence peptidase, neprilysin, angiotensin-converting enzyme, and combinations thereof. Suitable serine protease may be, for example, plasmin.

Other suitable proteases that may be attached to the support may be those containing a carboxylic acid group such as, for example, lipase, lysozyme, chymotrypsin, catalase, horseradish peroxidase, acetylcholinesterase, and combinations thereof. Still other suitable proteases that may be attached to the support may be, for example, casein. Yet other suitable proteases that may be attached to the support may be, for example, those containing a carboxyl group, amine group, hydroxyl group, or a polyethylene glycol (PEG) linker, and those that may be modulated through N-(3-dimethylaminopropyl)-N′-ethylcarbodiimide (EDC), N-hydroxysuccinimide (NHS) (“EDC/NHS”) chemistry.

Proteases may be recombinant proteases and proteases isolated and purified from tissue sources. Other suitable proteases may be portions of full-length proteases having only the active protease domain.

In another embodiment, the device has a therapeutically effective amount of at least one protease. As used herein, “a therapeutically effective amount” refers to that amount of protease that produces the desired effect and can be expected to vary with the mechanism of delivery, with mechanisms that provide greater access of proteases to proteins that are to be cleaved requiring a lesser amount. Suitable amounts may be, for example, that amount of protease that produces a reduction of protein in cerebrospinal fluid and/or brain interstitial fluid to 5% of their pre-treatment levels.

### Supports

Suitable supports may be, for example, electrospun fibers, electrosprayed fibers, microparticles, nanoparticles, polymers, ceramics, inorganic/polymer composites, and combinations thereof.

Electrospun Fiber Supports

Particularly suitable fiber supports include electrospun and electrosprayed nanofibers including at least one polymer and supporting at least one protease.

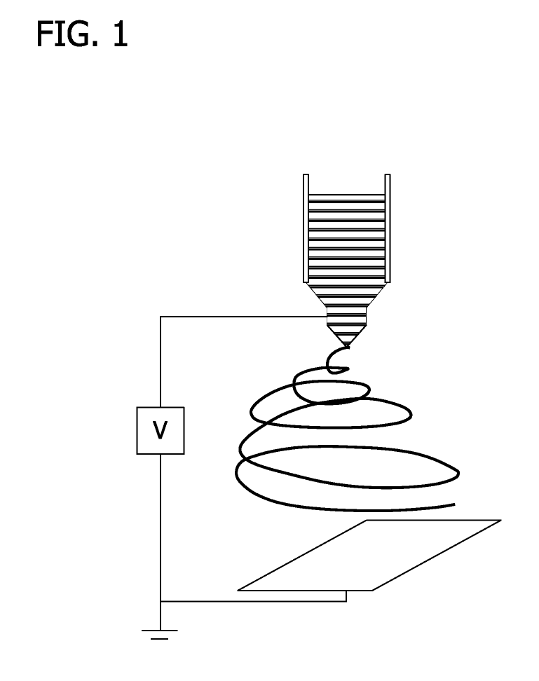

The electrospun fiber compositions may be made according to electrospinning methods known by those skilled in the art. Electrospinning generally uses an electric field to draw a solution including, for example, a polymer or ceramic, from the tip of nozzle to a collector. A high voltage DC current is applied to the solution that causes a jet of the solution to be drawn toward the grounded collector. Once ejected from the nozzle, the charged solution is evaporated to form fibers that are collected on the collector. FIG. 1 shows an example of an electrospinning apparatus that is suitable for preparing electrospun nanofiber supports of the present disclosure.

The size and morphology of the fibers depends on a variety of factors such as, for example, viscosity of the solution, molecular weight, nature of the polymer or ceramic, and parameters related to the electrospinning apparatus.

The fibers may be randomly oriented fibers and aligned fibers. The average fiber diameter may be, for example, from about 10 nm to about 100 p.m. Without being bound by theory, the smaller the diameter of the fiber, the higher the surface area the fiber may have. A higher surface area may permit higher concentrations of protease.

Suitable electrospun fiber compositions may be, for example, polymers and ceramics. Suitable polymer compositions may be, for example, polyesters and poly(phosphoesters). Particularly suitable substances for making electrospun and electrosprayed nanofibers may be, for example, acrylic, acrylic resin (96% acrylonitrile), blends of polyethylene-co-vinyl acetate and poly lactic acid, cellulose acetate, Nylon 6, Nylon 6 plus montmorillonite, poly (2-hydroxy ethyl methacrylate), poly (ether amide), poly (methyl methacrylate-random; PMMA-r-TAN), poly (p-phenylene terephthalamide), poly lactic acid, blends of polyaniline and polyoxide, polybenzimidazole, polycaprolactone, polycarbonate, polyether urethane, polyethylene, polyethylene naphthalate, polyethylene oxide, polyethylene terephthalate, polyethylene-co-vinyl acetate, poly-L-lactide, polypropylene, polystyrene, polyurethane, polyvinyl alcohol, polyvinyl chloride, polyvinylcarbazole, poly(vinyl-idene fluoride), poly(vinyl pyrrolidone), silk link polymer with fibronectin functionality (SLPF), styrene-butadiene-styrene, and combinations thereof.

Suitable nanofibers may also be those with other functional characteristics and properties. For example, nanofibers may be those with properties or compositions that differ on the outer surface or annulus from the inner core of the nanofiber. Other suitable nanofibers may be metal-containing and/or semiconducting nanofibers such as, for example, Pt(NH₂dmoc)₄(PtCl₄). Nanofibers may also be decorated or coated with nanoparticles. Nanofibers may further be chemically or optically treated to improve antimicrobial activity. Nanofibers may also be coated with metals such as, for example, nickel and gold.

Suitable nanofiber materials may be biodegradable, non-biodegradable, and combinations thereof. Particularly suitable biodegradable materials for making nanofibers may be, for example, poly(caprolactone), poly(L-lactide), poly(ethylene terephthalate), and poly(glycolide). Poly(ε-caprolactone), for example, is degradable over the course of approximately over two years. Polyglycolic acid, for example, is degradable over the course approximately two weeks. Suitable non-degradable materials may be, for example, polytetrafluoroethylene and polystyrene.

Particle Supports

Suitable supports for the present disclosure may also be particles. As used herein, particles also refer to beads and spheres. Particles may be microparticles and nanoparticles. As used herein, microparticle refers to particles having a mean particle size in the micrometer range. Suitable mean particle size of microparticles may be from about 1 μm to about 1,000 μm. More suitably, mean particle size of microparticles may be from about 2 μm to about 50 μm. As used herein, nanoparticle refers to particles having a mean particle size in the nanometer range. Suitable mean particle size of nanoparticles may be from about 1 nm to about 1000 nm. More suitably, nanoparticle size may be from about 50 nm to about 800 nm.

Suitable particles may be biodegradable and non-biodegradable. Suitable polymers for making particles may be poly-ε-caprolactone, polylactide, dermatan sulfate, chondroitin sulfate, keratin sulfate, heparin sulfate, dextran sulfate, chitosan, collagen, albumin, cellulose, gelatin, elastin, hyalauronic acid, polystyrene, polyacrylic acid, polyacrylonitrile, polyamide, polyacrylamide, polyacrolein, polybutadiene, polycaprolactone, polycarbonate, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, polysulfone, agar, metal, resin, latex, rubber, silicone, and combinations thereof.

Particles may be porous and non-porous. Suitable average pore size may be from about 0.1 nm to about 500 nm. Particles may also contain additional surface functional groups such as, for example, carboxylates, esters, alcohols, carbamides, aldehydes, amines, sulfur oxides, nitrogen oxides, or halides, to facilitate attachment of the protease to the particle as described in more detail below.

Other Supports

Support materials may also be biopolymers. Suitable biopolymers may be, for example, collagen and cellulose acetate.

Other suitable support may be ceramics and inorganic polymer composites. Suitable ceramics and inorganic polymer composites may be, for example, TiO₂/SiO₂, PbZrₓTi₁₋ₓO₃, SiO₂, indium tin oxide, GeO₂, NiFe₂O₄, BaTiO₃, Al₂O₃, CuO, NiO, V₂O₅, ZnO, Co₃O₄, Nb₂O₅, MoO₃, and MgTiO₃.

### Methods for Attaching Proteases to the Support

The support includes at least one protease. The protease is attached to the support by methods that are known to those skilled in the art. As used herein, the terms attached, immobilized, linked, coupled, connected, and/or affixed are used interchangeably to refer to the association between the protease and support.

The protease may be directly or indirectly attached to the support. Conjugation chemistry, for example, is a suitable process for directly attaching molecules to the support. As used herein, directly attached to the support refers to attaching the protease to the support by chemical methods. Non-covalent attachment is an indirect method for attaching proteases to the support. Additionally, proteases may be indirectly attached to the support by methods using linker or spacer molecules. Linker and spacer molecules may allow for additional distance or separation between the support and protease such that the support does not interfere, for example, by sterically hindering the activity of the protease with the protein. Suitable linker and spacer molecules may be, for example, chemical spacers, amino acid spacers, peptide spacers, or some combination thereof.

Particularly suitable methods for attaching the protease are described in Stoilova et al. (Eur. Polymer J. 46:1966-1974 (2010)), Reichmuth et al. (Bioconjugate Chem. 13:90-96 (2002)), Ma et al. (Biomaterials 26:2527-2536 (2005)), Wang et al. (Biomacromolecules 3:1286-1295 (2002)), which are hereby incorporated by reference in their entireties.

The protease may be reversibly or irreversibly attached to the support. If the protease is reversibly attached to the support, the protease can become unassociated with the support due to diffusion away from the support. In another embodiment, the protease that is reversibly attached the support may become unassociated with the support due to degradation of the support. A protease that may be irreversibly attached to a support may be covalently or non-covalently attached to the support such that the protease cannot diffuse away from the support.

### External (Ex Vivo) Devices

In another aspect, the present disclosure is directed to an externally positioned device wherein cerebrospinal fluid and/or brain interstitial fluid is treated in an active flow system. For example, the device may operate as an ex vivo system wherein cerebrospinal fluid and/or brain interstitial fluid is withdrawn from a subject by a catheter, circulated through the device wherein the cerebrospinal fluid and/or brain interstitial fluid contacts the support and protease, and is then returned to the subject via a catheter. Circulation of the cerebrospinal fluid and/or brain interstitial fluid may be accomplished by methods known in the art. For example, the system may include a peristaltic pump to circulate cerebrospinal fluid and/or brain interstitial fluid through the catheter and device.

Cerebrospinal fluid and/or brain interstitial fluid may be withdrawn from the subject by a catheter positioned in a ventricle, a subarachnoid space, cisterna magna, or intrathecal (i.e., intradural) space in the cervical, thoracic, or lumbar spine. The cerebrospinal fluid and/or brain interstitial fluid enters the device where it contacts the device including the support and at least one protease. Proteases attached to the support will cleave protein in the cerebrospinal fluid and/or brain interstitial fluid. After passing through the device, the resultant cerebrospinal fluid and/or brain interstitial fluid may be returned to the subject via another catheter. The return catheter may be located in a similar position as the catheter used to withdraw the cerebrospinal fluid and/or brain interstitial fluid. Alternatively, the catheter used to return the cerebrospinal fluid and/or brain interstitial fluid may be located in a different position than the catheter used to withdraw the cerebrospinal fluid and/or brain interstitial fluid. In still another alternative, a dual-lumen catheter may be used to both withdraw and return cerebrospinal fluid and/or brain interstitial fluid to the subject.

In another active flow system embodiment, the device may be implanted as an in vivo system. The device may be implanted in any location determined by those skilled in the art. For example, the device may be subcutaneously implanted, intramuscularly implanted, intraabdominally implanted, intracranially implanted (extradurally, intraudrally, intraparenchymally, or intraventriculary) placed as determined by those skilled in the art. The device may also be implanted in any anatomical location such as, for example, the lumbar region, intracranially, extracranially/subcutaneously, in a space within the calvarium of the skull, intrabdominally, subcutaneously over the back, or in the abdomen. Catheters are connected to a pump to circulate cerebrospinal fluid and/or brain interstitial fluid through the device where it is contacted with a support having a protease attached as previously described. The treated cerebrospinal fluid and/or brain interstitial fluid may then be returned to the subject.

In another implantable active flow system embodiment, the device may be an implantable reservoir that may be periodically accessed. Catheters connected to a pump circulate cerebrospinal fluid and/or brain interstitial fluid through the device where it is contacted with a support having a protease attached as previously described. The treated cerebrospinal fluid and/or brain interstitial fluid may then be returned to the subject.

In yet another implantable active flow system embodiment, the device having the support and attached protease may be built into a catheter system. A pump may also be built into the catheter system to circulate cerebrospinal fluid and/or brain interstitial fluid.

In another embodiment, the device may be an externally located device wherein cerebrospinal fluid and/or brain interstitial fluid may be treated in a passive system. In such an embodiment, cerebrospinal fluid and/or brain interstitial fluid may be removed from a subject and transferred to a reservoir holding a device that includes a support and at least one protease attached to the support. The reservoir may be any type of container such as, for example, a tube, syringe, jar, beaker, canister, and flask. While in the reservoir, proteins in the cerebrospinal fluid and/or brain interstitial fluid are cleaved by proteases attached to the support. Following a period of incubation, treated cerebrospinal fluid and/or brain interstitial fluid may then be returned to the subject.

Suitable locations for removing cerebrospinal fluid and/or brain interstitial fluid may be, for example, from a cerebral ventricle, lumbar area, and the cisterna magna. The cerebrospinal fluid and/or brain interstitial fluid may be returned to the subject in the same location or a different location from which the cerebrospinal fluid and/or brain interstitial fluid is removed. In one embodiment, the cerebrospinal fluid and/or brain interstitial fluid may be removed from and returned to the same location by, for example, a double-lumen catheter.

In another embodiment, the device may further include a housing. The housing may be constructed of any material depending on the intended use. If, for example, the device is to be used as an ex vivo active flow system, the housing material may be constructed of any materials that provide protection and seal the device including the support and attached protease such that cerebrospinal fluid and/or brain interstitial fluid remains contained within the system. If, for example, the device is to be implanted as an in vivo system, the housing should be constructed of a material that is compatible with the subject so as to not cause an adverse reaction such as, for example, an immune response.

Catheters direct cerebrospinal fluid and/or brain interstitial fluid from the subject through an input of the device such that the cerebrospinal fluid and/or brain interstitial fluid contacts the device including the support and at least one protease attached. An output from the device is connected to catheter to return the treated cerebrospinal fluid and/or treated brain interstitial fluid to the subject.

An external (ex vivo) device may be constructed of any material such as stainless steel, plastic, glass, and other materials. The device including the support and at least one protease may be contained by the housing. If, for example, the housing is a glass or plastic column and the support is particles or beads having protease attached, the particles or beads may be packed in the column and cerebrospinal fluid and/or brain interstitial fluid may be applied to the column and collected as it flows out of the column. The collected treated cerebrospinal fluid and/or brain interstitial fluid may then be returned to the subject. If, for example, the housing is a canister and the support is a nanofiber mat, the nanofiber mats may be contained in another housing such as, for example, a baffle that is held in the housing. The housing may hold more than one baffle and each baffle may hold one more nanofiber mat.

The housing may include other components. If, for example, the device is used for an ex vivo active flow system the circulation mechanism and/or other components may be separate from the device including the support and protease attached to the support. Circulation may be provided by an external circulation mechanism such as, for example, a peristaltic pump. If, for example, the device is used in an implantable (in vivo) active flow system the housing may also hold internal components such as the circulation mechanism, a power system (e.g., batteries), and other components. Cerebrospinal fluid and/or brain interstitial fluid may enter and exit the housing through inputs and outputs as previously described. Following contact with the device including the support, the treated cerebrospinal fluid and/or treated brain interstitial fluid may be returned to the subject.

In other embodiments, the device including the support and at least one protease attached to the support may be contained within an additional housing or structure. For example, nanofiber mats or particles including the protease may be housed in a cartridge that may be placed into a larger housing such that cartridges may be replaced while leaving the larger housing undisturbed. Nanofiber mats may also be housed in a frame-like structure, baffles, cages, cubes, and spheres. The housing should allow the cerebrospinal fluid and/or brain interstitial fluid to be contacted with the support and the protease. Catheters direct cerebrospinal fluid and/or brain interstitial fluid to the support held by the cartridge such that the cerebrospinal fluid and/or brain interstitial fluid comes in contact with the protease attached to the support as described herein.

Optionally, cerebrospinal fluid and/or brain interstitial fluid may be further processed before and/or after it is contacted with the device. For example, filtering the cerebrospinal fluid and/or brain interstitial fluid after it is contacted with the device and before it is returned to the subject may be performed to remove cleaved protein fragments. Cerebrospinal fluid and/or brain interstitial fluid may also be analyzed before and/or after it is contacted with the device. For example, cerebrospinal fluid and/or brain interstitial fluid may be analyzed by, for example, gel electrophoresis, Western blot analysis, ELISA, HPLC, mass spectrometry, and other analytical methods, before and after contact with the device to monitor alteration, reduction, and/or concentration of protein.

### Administration

In another aspect, a device of the present disclosure may be administered locally to a desired location having cerebrospinal fluid and/or brain interstitial fluid. For example, in one embodiment, the device may be administered into the subarachnoid space. In yet another embodiment, the device may be introduced intrathecally such as, for example, a cerebral ventricle, lumbar area, or the cisterna magna. It may be placed intraparenchymally in a region of interest or it may be laid over the cortical surface of the brain which could include the frontal lobes, the temporal lobes, the parietal lobes, the occipital lobes. The device may also be placed over and/or in various folds or sulci of the brain such as the sylvian fissure, the central sulcus, the ambien cistern (adjacent to the hippocampus), the hippocampul sulcus, interhemispheric fissure, and the like, as determined by those skilled in the art.

In another embodiment, the device may be directly implanted into the subject to function as an in vivo passive system. For example, if the device is a nanofiber mat, a particle, or a particle-conjugated nanofiber mat as described herein, the nanofiber mat, particles, or particle-conjugated nanofiber mat may be implanted into a ventricle of the brain, spinal column, the cisterna magna, the subarachnoid space, or in the substance of the brain itself where cerebrospinal fluid and/or brain interstitial fluid may contact the device and result in cleavage of proteins as they come in contact with the protease attached to the nanofiber mat, particles, or particle-conjugated nanofiber mat.

In embodiments wherein the support is made from a biodegradable material, the protease may further become unassociated with the support as the material degrades. Unassociated protease may diffuse into the cerebrospinal fluid and/or brain interstitial fluid and cleave proteins. Biodegradable supports may be used to deliver proteases into the cerebrospinal fluid and/or brain interstitial fluid in a time-release manner. Time-release of the proteases may be determined according to the selection of support material as known by those skilled in the art.

Methods for Altering Protein in Cerebrospinal Fluid and/or Brain Interstitial Fluid

In another aspect, the present disclosure is directed to methods for altering protein in cerebrospinal fluid and/or brain interstitial fluid. The method includes contacting cerebrospinal fluid and/or brain interstitial fluid with a support. The support includes at least one protease attached to the support. The protease cleaves protein in cerebrospinal fluid and/or brain interstitial fluid resulting in the alteration of protein. As used herein, alteration of protein refers to cleavage of protein that results in the reduction or decrease in the amount, level, and/or concentration of the protein in the cerebrospinal fluid and/or brain interstitial fluid. Alteration may also result in the increase in concentration due to the increase in peptides resulting from cleavage by the protease. As used herein, reduction of protein refers to the decrease in the amount, level and/or concentration of protein in cerebrospinal fluid and/or brain interstitial fluid as compared to cerebrospinal fluid and/or brain interstitial fluid that has not received treatment involving a support having at least one protease attached. Concentration of protein may be determined by methods known by those skilled in the art. For example, concentration may be determined by protein assays, gel electrophoresis and densitometry, and chromatography methods (e.g., HPLC).

Suitable supports may be any support as previously described. A particularly suitable support is an electrospun nanofiber support. Suitable proteases may be any protease as previously described.

The method is suitable for altering any protein in cerebrospinal fluid and/or brain interstitial fluid. Particularly suitable proteins for which the method is suitable are amyloid beta, tau, α-synuclein, prion protein, polyglutamine-containing proteins, an antibody, superoxide dismutase 1, anti-cardiolipin antibody, an anti-myelin antibody, and combinations thereof.

In one aspect, the method includes contacting cerebrospinal fluid and/or brain interstitial fluid in an active flow system as previously described. In another aspect, the method includes contacting cerebrospinal fluid and/or brain interstitial fluid in a passive system as previously described.

In one embodiment, the method includes contacting cerebrospinal fluid and/or brain interstitial fluid in an ex vivo active flow system. In another embodiment, the method includes contacting cerebrospinal fluid and/or brain interstitial fluid in a passive system whereby cerebrospinal fluid and/or brain interstitial fluid is removed from a subject and placed in a reservoir having a device including a support and at least one protease attached. Following an incubation period, treated cerebrospinal fluid and/or brain interstitial fluid may then be returned to the subject.

In another embodiment, the method includes contacting cerebrospinal fluid and/or brain interstitial fluid in an in vivo system whereby protein is altered through physiologic flow of cerebrospinal fluid and/or brain interstitial fluid over a stationary device including a support and at least one protease attached to the support. The device may be positioned intraventricularly or subdurally, and through associated diffusive processes.

In another embodiment, the method includes contacting cerebrospinal fluid and/or brain interstitial fluid in an in vivo active flow system. In another embodiment, the method includes contacting cerebrospinal fluid and/or brain interstitial fluid in an in vivo passive system whereby a device including a support and at least one protease attached to the support is administered to a subject.

The method may further include additional treatment of cerebrospinal fluid and/or brain interstitial fluid before returning it to the subject. For example, cerebrospinal fluid and/or brain interstitial fluid may be filtered after it is contacted with the device to remove cleaved protein fragments. Cerebrospinal fluid and/or brain interstitial fluid may also be analyzed before being returned to the subject. For example, cerebrospinal fluid and/or brain interstitial fluid may be analyzed by, for example, gel electrophoresis, Western blot analysis, ELISA, HPLC, mass spectrometry, and other analytical methods, before and after contact with the device to monitor reduction of protein.

### Methods for Treating Neurodegenerative Diseases

In another aspect, the present disclosure is directed to methods for treating neurodegenerative diseases. Suitable neurodegenerative diseases may be, for example, Alzheimer's disease, Parkinson's disease, Prion disease, Polyglutamine disease, Tauopathy, and Familial amyotrophic lateral sclerosis.

The method includes contacting cerebrospinal fluid with a device. The device includes a support and at least one protease attached to the support, wherein the protease cleaves a protein associated with the disease. For example, amyloid beta is associated with Alzheimer's disease. Thus, amyloid beta including amyloid beta oligomers and plaques are cleaved according to the method. Tau tangles are also associated with Alzheimer's disease and Tauopathy, for example, and may be cleaved according to the method. Parkinson's disease, for example, is associated with Lewy bodies formed by α-synuclein. Prion disease, for example, is associated with PrP^{Sc} and prion plaques. Polyglutamine disease, for example, is associated with polyglutamine-containing proteins. Familial amyotrophic lateral sclerosis, for example, is associated with superoxide dismutase 1 and Bunina bodies. Thus, suitable proteins may be, for example, amyloid beta, including amyloid oligomers and plaques, tau, including tau tangles, α-synuclein, prions, including prion plaques, polyglutamine-containing proteins, superoxide dismutase, including Bunina bodies, and combinations thereof.

Suitable supports may be any support as previously described. A particularly suitable support is an electrospun nanofiber support. Suitable proteases may be any protease as previously described.

In one aspect, the method includes contacting cerebrospinal fluid and/or brain interstitial fluid in an active flow system as previously described. In another aspect, the method includes contacting cerebrospinal fluid and/or brain interstitial fluid in a passive system as previously described.

### Methods for Treating Acute Neurological Disorders

In another aspect, the present disclosure is directed to methods for treating acute neurological disorders. Suitable neurological disorders may be, for example, acute demyelinating disorder and systemic lupus erythematosus.

The method includes contacting cerebrospinal fluid and/or brain interstitial fluid with a device. The device includes a support and at least one protease attached to the support, wherein the protease cleaves a protein associated with the disease. A particularly suitable protein that is cleaved by the protease may be, for example, an anti-myelin antibody.

Suitable supports may be any support as previously described. A particularly suitable support is an electrospun nanofiber support. Suitable proteases may be any protease as previously described.

In one aspect, the method includes contacting cerebrospinal fluid and/or brain interstitial fluid in an active flow system as previously described. In another aspect, the method includes contacting cerebrospinal fluid and/or brain interstitial fluid in a passive system as previously described.

### Methods for Treating Alzheimer's Disease

In another aspect, the present disclosure is directed to methods for treating Alzheimer's disease. The method includes contacting cerebrospinal fluid and/or brain interstitial fluid with a device. The device includes a support and at least one protease attached to the support, wherein the protease cleaves amyloid beta, including amyloid beta oligomers and plaques. The protease may further cleave tau protein and tau tangles.

Suitable supports may be any support as previously described. A particularly suitable support is an electrospun nanofiber support. Suitable proteases may be any protease as previously described.

In one aspect, the method includes contacting cerebrospinal fluid and/or brain interstitial fluid in an active flow system as previously described. In another aspect, the method includes contacting cerebrospinal fluid and/or brain interstitial fluid in a passive system as previously described.

In one embodiment, the method includes contacting cerebrospinal fluid and/or brain interstitial fluid in an ex vivo active flow system. In another embodiment, the method includes contacting cerebrospinal fluid and/or brain interstitial fluid in a passive system whereby cerebrospinal fluid is removed from a subject and placed in a reservoir having a device including a support and at least one protease attached. Following an incubation period, treated cerebrospinal fluid is returned to the subject.

In another embodiment, the method includes contacting cerebrospinal fluid and/or brain interstitial fluid in an in vivo system whereby protein is altered by the reduction of protein concentration by physiologic flow of cerebrospinal fluid and/or brain interstitial fluid over a stationary device including a support and at least one protease attached that is positioned intraventricularly or subdurally, and through associated diffusive processes. Without being bound by theory, it is believed that as protein concentrations decrease in cerebrospinal fluid that has contacted the device, protein will diffuse into the cerebrospinal fluid having reduced protein, thereby altering protein concentrations throughout the system and potentially dissolving plaques. Additionally, brain interstitial fluid, which exists in equilibrium with cerebrospinal fluid, will be drawn out of interstitial spaces by normal circulation where proteins within the interstitial space will then be drawn into circulation and available to contact the device.

In another embodiment, the method includes contacting cerebrospinal fluid and/or brain interstitial fluid in an in vivo active flow system. In another embodiment, the method includes contacting cerebrospinal fluid and/or brain interstitial fluid in an in vivo passive system whereby a device including a support and at least one protease attached to the support is administered to a subject.

The method may further include additional treatment of cerebrospinal fluid before returning it to the subject. For example, cerebrospinal fluid may be filtered after it is contacted with the device to remove cleaved protein fragments. Cerebrospinal fluid may also be analyzed before being returned to the subject. For example, cerebrospinal fluid may be analyzed by, for example, gel electrophoresis, Western blot analysis, ELISA, HPLC, mass spectrometry, and other analytical methods, before and after contact with the device to monitor alteration of protein.

Without being bound by theory, it is believed that small peptide fragments resulting from cleavage of proteins are no longer physiologically or pathologically active and will be naturally resorbed from the cerebrospinal fluid through normal cerebrospinal fluid turnover by being absorbed into venous system via the arachnoid granulations. Thus, with amyloid beta, for example, large amyloid protein oligomers (of 10 kDa and greater molecular weight) as well as smaller amyloid protein (of 5 kDa) will be cleaved to fragments less than 5 kDa such that harmful amyloid beta protein is scrubbed from the cerebrospinal fluid. It is also believed that proteases such as, for example, neprilysin are native proteases that will naturally degrade.

It is further believed that the devices and method of the present disclosure may create a concentration gradient of protein in cerebrospinal fluid that may result in mass transit of protein from the interstitial fluid of the central nervous system into the cerebrospinal fluid, thus resulting in a total reduction of the protein in the brain substance. Thus, with amyloid beta, for example, the reduction of amyloid beta by the device creates a concentration gradient of amyloid beta into the cerebrospinal fluid. As the cerebrospinal fluid having reduced amyloid beta naturally circulates within the subject, this may, in turn, draw amyloid beta from interstitial spaces into the cerebrospinal fluid because of the concentration gradient. Once in the cerebrospinal fluid, the protein may contact the device and be cleaved by the protease attached to the support. The result is an overall reduction in amyloid beta.

The disclosure will be more fully understood upon consideration of the following non-limiting Examples.

### EXAMPLES

### Example 1

In this Example, nanofibrous neprilysin mats are prepared.

### Preparation of Electrospun Fiber Mats

Nanofibers were generated by electrospinning using a setup that was modified from earlier studies (Xie et al., Macromol. Rapid Commun. 29:1775 (2008); Xie et al., Biomaterials 30:354 (2009), which are herein incorporated by reference in their entireties). Poly(ε-caprolactone) (PCL; MW 65,000 g/mol; Sigma-Aldrich, St. Louis, Mo.) was dissolved in a solvent mixture of dichloromethane and N,N-dimethylformamide (Fisher Chemical, Waltham, Mass.) with a ratio 8:2 (v/v) at a concentration of 20% (w/v). Polymer solution was pumped at a flow rate of 0.2 mL/hour using a syringe pump. A DC high voltage of 12 kV was applied between a nozzle (22-gauge needle) and a rotating mandrel covered with an aluminum foil for use as the grounded collector. FIG. 1 shows the electrospinning set up. Fibers deposited on the aluminum foil were peeled off and fiber membranes were treated with air plasma cleaner for 20 minutes.

### Conjugation of Neprilysin to Electrospun Nanofiber Mats

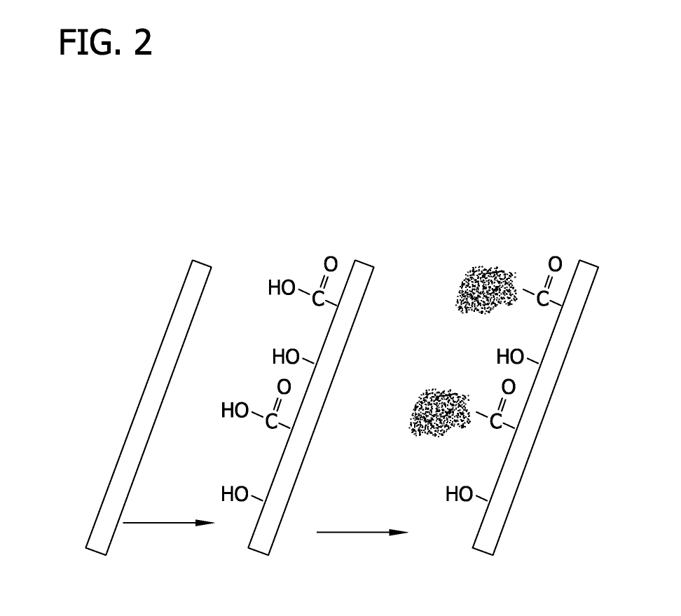

Oxygen containing groups (i.e., carboxylic acid and hydroxyl) were developed on electrospun nanofiber mats during the air plasma treatment. Subsequently, N-(3-dimethylaminopropyl)-N′-ethylcarbodiimide (EDC), N-hydroxysuccinimide (NHS) and neprilysin in PBS were added to the fibers and incubated at 4° C. for 24 hours. Neprilysin was immobilized on the PCL nanofibers due to EDC/NHS crosslinking by conjugation of carboxyl groups to amine groups. FIG. 2 is a schematic illustrating EDC/NHS-mediated attachment of neprilysin to air plasma treated nanofibers. Neprilysin-bound nanofiber membranes were washed with PBS three times before use.

The morphology and structures of various fiber assemblies were characterized by scanning electron microscopy (SEM) (200 NanoLab, FEI, Oregon). To avoid charging, the polymer fiber samples were coated with platinum using a sputter coater for 40 seconds in vacuum at a current intensity of 40 mA after the sample was fixed on a metallic stud with double-sided conductive tape.

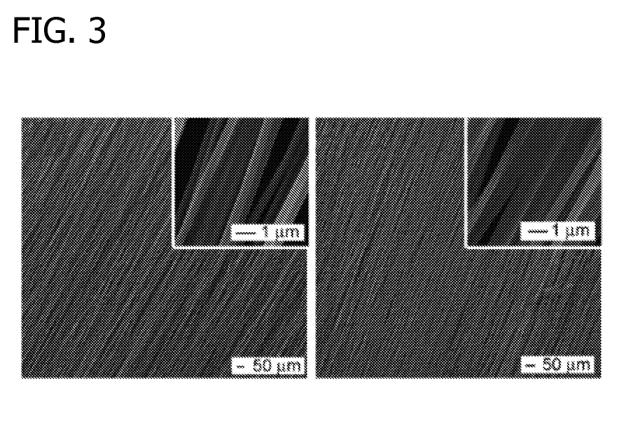

FIG. 3 shows SEM images of nanofibers before (A) and after (B) air plasma treatment (accelerating voltage of 15 kV). Insets show enlarged views of nanofibers.

### Example 2

In this Example, neprilysin nanofiber mats were tested for the ability to reduce amyloid beta protein from simulated cerebrospinal fluid (“artificial CSF) in an active flow system.

Specifically, an artificial cerebrospinal fluid was prepared containing the following: NaCl (124 mM); KCl (2 mM); KH₂PO₄ (1.25 mM); MgSO₄ (0.5 mM); CaCl₂ (2 mM); NaHCO₃ (26 mM). Amyloid beta protein (1.1 ng/mL) was added to the artificial CSF. A system containing a reservoir connected in series with a peristaltic pump and a canister containing neprilysin nanofiber mats was constructed and flushed with a solution of bovine serum albumin to block amyloid beta interaction with surfaces. 400 mL of the artificial CSF/amyloid beta solution was placed in a reservoir to represent an approximate daily quantity of CSF and amyloid beta production in an adult human. The reservoir was placed on a shake table set to promote uniform concentration of solutes and the pump was set to 180 RPM. Samples were periodically collected from the reservoir and analyzed by gel electrophoresis (18% Tris-Glycine polyacrylamide gels).

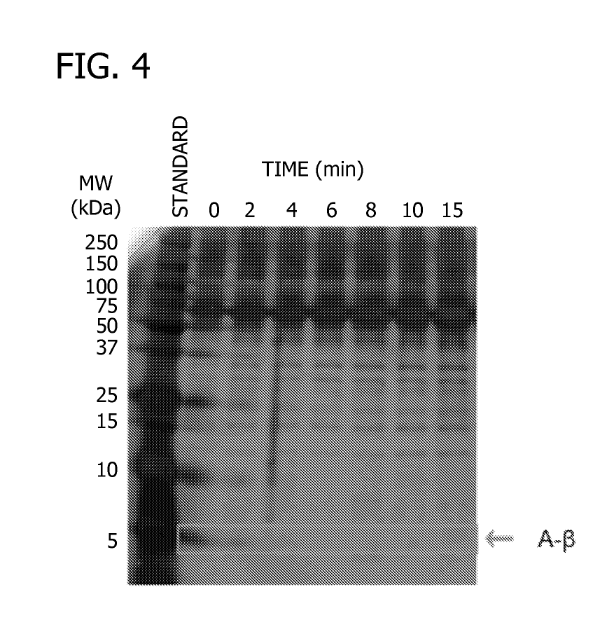

As shown in FIG. 4, silver-stained gels indicated the strong initial presence of a ˜5 kDa protein, consistent with the molecular weight of amyloid beta, at time 0 that rapidly decreased below detectable concentrations after 4 minutes of fluid circulation. Additionally, bands having molecular weights of 10 kDa and 25 kDa, which are believed to be amyloid beta oligomers, also decreased over time. These results demonstrate that neprilysin attached to the neprilysin nanofiber mat maintains its activity and can decrease amyloid beta levels in CSF.

### Example 3

In this Example, neprilysin nanofiber mats were tested for the ability to reduce amyloid beta protein from artificial cerebrospinal fluid in a passive system.

A plastic tube was flushed with a solution of BSA to block nonspecific binding of amyloid beta. The tube was then filled with 50 mL of artificial CSF/amyloid beta, as described above. A 1-inch neprilysin nanofiber mat was inserted into the solution and the tube was placed on a shake table. Samples were periodically collected and analyzed by gel electrophoresis as described above.

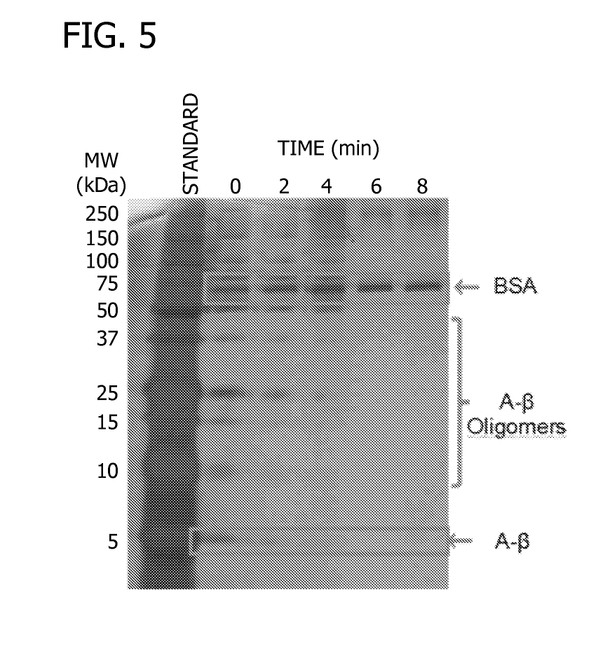

As shown in FIG. 5, the silver-stained gel indicated the strong initial presence of a ˜5 kDa protein, consistent with the molecular weight of amyloid beta, at time 0 that rapidly decreased below detectable concentrations after 2 minutes of fluid incubation. Additionally, bands having molecular weights of 10 kDa, 25 kDa, and 37 kDa, which are believed to be amyloid beta oligomers, also decreased over time. The decreased clearance time of the passive system as compared to the active flow system was attributed to the smaller volume of artificial CSF/amyloid beta solution.

### Example 4

In this Example, tests will be performed to determine whether neprilysin nanofiber mats can reduce amyloid beta in an animal model.

Specifically, mice expressing a chimeric mouse/human amyloid precursor protein (Mo/HuAPP695swe) and a mutant human presenilin 1 (PS1-dE9) will be obtained from Jackson Laboratory (Bar Harbor, Me.). Mice will be treated by implanting neprilysin nanofiber mats. Neprilysin-conjugated nanofiber mats (approximately 0.08 inch wide) will be rolled up and slid into 26-gauge stainless steel tubes of a bilateral pedestal prior to implantation. Coordinates will be selected for the area of maximal cross-sectional diameter of the lateral ventricles, approximately −0.5 mm Bregma. Once in place, a dummy cannula will be inserted into the pedestal, forcing the neprilysin nanofiber mat from the tube into the ventricular space. The cannula will then be capped and the entire pedestal will be sealed in place with Cerebond Skull Fixative.

Three groups of 20 mice will be used for this experiment. One group will be implanted with neprilysin nanofiber mats, one group will be implanted with nanofiber mats without attached neprilysin, and one group will be subjected to the implantation protocol using an empty pedestal/cannula system. Mice will be maintained for a length of time prescribed in part by the lifespan of the nanofiber mat as determined previously, but not more than three months.

Four mice from each study group will be sacrificed at one day, one week, one month, and after three months for assessment of encapsulation and plaque load. Amyloid plaque formation will be assessed by stained slides of the mouse brains obtained at the time points.

Results are expected to demonstrate that neprilysin nanofiber mats implanted into ventricles of mice will arrest amyloid plaque formation without an undue rise in morbidity by reducing free amyloid beta levels in the cerebrospinal fluid.

1.-34. (canceled)

35. A device for altering protein in cerebrospinal fluid and/or brain interstitial fluid, the device comprising:
a support; and
a protease attached to the support, wherein the protease is selected from the group consisting of a zinc metalloprotease, a serine protease, trypsin, pepsin, and combinations thereof.

36. The device of claim 35, wherein the zinc metalloprotease is selected from the group consisting of insulin degrading enzyme, presequence peptidase, neprilysin, angiotensin-converting enzyme, and combinations thereof.

37. The device of claim 35, wherein the serine protease is plasmin.

38. The device of claim 35, wherein the support is selected from the group selected of a fiber, a membrane, a gel, a particle, and combinations thereof.

39. The device of claim 35, wherein the support is biodegradable or non-biodegradable.

40. The device of claim 35, wherein the protease is directly attached to the support or indirectly attached to the support.

41. A method for altering protein in a bodily fluid, the method comprising:
contacting a bodily fluid selected from the group consisting of cerebrospinal fluid and brain interstitial fluid with a device, wherein the device comprises a support and a protease attached to the support, wherein the protease is selected from the group consisting of a zinc metalloprotease, a serine protease, trypsin, pepsin, and combinations thereof, and wherein the protease cleaves at least one protein in the bodily fluid.

42. The method of claim 41, wherein the zinc metalloprotease is selected from the group consisting of neprilysin, insulin degrading enzyme (insulysin), presequence peptidase, angiotensin-converting enzyme, and combinations thereof.

43. The method of claim 41, wherein the protein is amyloid beta, tau, α-synuclein, prion protein, polyglutamine-containing proteins, superoxide dismutase 1, an antibody, an anti-cardiolipin antibody, an anti-myelin antibody, and combinations thereof.

44. The method of claim 41, wherein the serine protease is plasmin.

45. The method of claim 41, wherein the support is selected from the group consisting of a fiber, a membrane, a gel, a particle, and combinations thereof.

46. The method of claim 41, wherein the bodily fluid is re-introduced into a subject after contacting with the support.

47. The method of claim 41, wherein the support is contained in a housing.

48. The method of claim 41, further comprising implanting the device in a subject.

49. A method for treating a neurological disease or disorder, the method comprising:
contacting a bodily fluid selected from the group consisting of cerebrospinal fluid and brain interstitial fluid with a device, wherein the device comprises a support and a protease attached to the support, wherein the protease is selected from the group consisting of a zinc metalloprotease, a serine protease, trypsin, pepsin, and combinations thereof, and wherein the protease cleaves at least one protein in the bodily fluid.

50. The method of claim 49, wherein protein concentration in the bodily fluid is reduced as compared to a sample of the bodily fluid not contacted with a support comprising a protease attached to the support, wherein the protease is selected from the group consisting of a zinc metalloprotease, a serine protease, trypsin, pepsin, and combinations thereof.

51. The method of claim 49, wherein the at least one protein is selected from the group consisting of amyloid beta, tau, α-synuclein, prion protein, polyglutamine-containing proteins, superoxide dismutase 1, an antibody, an anti-cardiolipin antibody, an anti-myelin antibody, and combinations thereof.

52. The method of claim 49, wherein the neurological disease or disorder is selected from the group consisting of Alzheimer's disease, Parkinson's disease, Prion disease, Polyglutamine disease, Tauopathy, Familial amyotrophic lateral sclerosis, an acute demyelinating disorder and systemic lupus erythematosus.

53. The method of claim 49, wherein the support is selected from the group consisting of a fiber, a membrane, a gel, a particle, and combinations thereof.

54. The method of claim 49, wherein the device is implanted in a peritoneal space, a subarachnoid cavity, brain parenchyma, spine parenchyma, a brain ventricle, cisterna magna, extrancranially in the head, subcutaneously in the body, in the calvarium of the skull, intrathecally in the spine and combinations thereof.
